(19) United States
(12) Patent Application Publication
(10) Pub. No.: US 2024/0149035 A1
(43) Pub. Date: May 9, 2024

(54) Conductive Biocompatible Scaffold For Electroporation

(71) Applicants: Ana Elena Kadala, Union City, CA (US); Fibralign Corporation, Union City, CA (US); ConductiveBio Incorporated, Santa Clara, CA (US)

(72) Inventors: Ana Elena Kadala, Houston, TX (US); Michael Paukshto, Foster City, CA (US); John Cooke, Houston, TX (US); Michael Spaid, Mountain View, CA (US)

(21) Appl. No.: 18/279,853

(22) PCT Filed: Mar. 1, 2022
(86) PCT No.: PCT/US2022/018393

§ 371 Date: Aug. 31, 2023

Related U.S. Application Data
(60) Provisional application No. 63/155,216, filed on Mar. 1, 2021.

(51) Int. Cl. A61M 37/00; A61N 1/32

(52) U.S. Cl. CPC A61M 37/0092; A61N 1/327

(57) ABSTRACT

We describe an improved conductive biocompatible scaffold for electroporation. The biocompatible scaffold comprises biocompatible material (e.g., collagen and/or other extracellular matrix) and incorporates a metal or polymer network or dispersion that conducts an electrical current through the scaffold. The material can adsorb or trap or bind nucleic acids (e.g., RNA or DNA), nanoparticles, proteins and/or small molecules; subsequently cells are placed in proximity to or in contact with the scaffold and an electrical current passed through the scaffold, thereby facilitating cellular entry of the nucleic acids, nanoparticles, proteins and/or small molecules, particularly into those cells that are in proximity of the scaffold.

### CROSS-REFERENCE TO RELATED APPLICATIONS

This application claims the benefit of, and priority to, U.S. Provisional Application No. 63/155,216 filed on Mar. 1, 2021, which is hereby incorporated by reference in its entirety.

### FIELD

The present disclosure (invention) relates broadly to the field of conductive biocompatible materials and structures for electroporation or magnetoporation. More specifically, embodiments of the present disclosure relate to a methods and devices, such as conductive biocompatible scaffolds, for electroporation comprising biocompatible materials (such as collagen and/or other biopolymers and/or extracellular matrix) that incorporate metal or conductive polymer networks that are above or below the percolation threshold, or dispersions that conduct an electrical current and facilitate or enhance permeation of a cell membrane, allowing chemicals, drugs, or nucleic acids to be introduced into a cell located in a vicinity of the conductive network.

### BACKGROUND

Shocking mammalian cells with an electromagnetic field, known as electroporation, was developed to effectively transfer nucleic acids into cells. Direct observation showed that an elevated electromagnetic field creates cell membrane instability and pores in the cell membrane through which small molecules diffuse. The type of molecule being introduced, the magnitude of the field applied, the length of the electromagnetic pulse, the buffer solution (media environment), and the type of cells used are important parameters that yield reversible permeabilization of the cell membrane and effective cargo delivery. Once the cells are electroporated, these variables can be altered or modified to facilitate closure of the cell membrane pores, which preserves the viability of the transfected cells.

Here we will interpret electroporation in a broad sense, including in this concept the excitation and/or stimulation of cells by an electric or magnetic field.

Various electroporation techniques have been developed. One conventional electroporation method is shown in FIG. 1 and utilizes the two parallel electrodes to induce an electric field in a cell solution between the electrodes. Thus, the cells can be introduced in a special cell media or plated on a scaffold, see, e.g., Brun P, Dettin M, Campana L G, Dughiero F, Sgarbossa P, Bernardello C, Tosi A L, Zamuner A, Sieni E. Cell-seeded 3D scaffolds as in vitro models for electroporation. Bioelectrochemistry. 2019. 125:15-24.

Another conventional electroporation method utilizes a porous substrate (insulator) as shown generally in FIG. 2 and further described by Cao Y, Ma E, Cestellos-Blanco S, Zhang B, Qiu R, Su Y, Doudna J A, Yang P. Nontoxic nanopore electroporation for effective intracellular delivery of biological macromolecules. Proc Natl Acad Sci USA. 2019 04 16; 116(16):7899-7904.

Conventional electroporation techniques have also used patterned electrodes as generally shown in FIG. 3 and further described by Santra T S and Tseng F G. Recent Trends on Micro/Nanofluidic Single Cell Electroporation. Micromachines 2013, 4, 333-356.

While various electroporation techniques have been developed all existing methods have: relatively low cell viability (typically, 20-50%) and low transfection rate (less than 50% in mammalian cell lines and much less in hard-to-transfect cells).

Accordingly, new developments and innovations are needed.

### SUMMARY

Embodiments of the present disclosure describe improved conductive devices and methods for electroporation over the current methods of cellular electroporation. In one embodiment, a device is provided that generally includes a biocompatible scaffold comprised of biocompatible material (e.g., collagen and/or other extracellular matrix, and the like) and incorporates metal or polymer networks that are either above or below the percolation threshold, or dispersions of conductive particles that provide an electrical current through the scaffold. The biocompatible materials that make up the scaffold are configured to adsorb or trap or bind nucleic acids (e.g., RNA or DNA), nanoparticles, proteins and/or small molecules. Subsequently, cells are placed in proximity to or in (direct) contact with the scaffold and an electrical current is passed through the scaffold, thereby facilitating cellular entry of the nucleic acids, nanoparticles, proteins and/or small molecules, particularly into those cells that are in proximity of the scaffold.

In some embodiments the scaffold comprises an aligned collagen material that induces an alignment of adherent cells. The orientation of the electric field with respect the cell alignment direction can be further optimized as presented below.

In some embodiments the nucleic acids, nanoparticles, proteins and/or small molecules can be introduced to the scaffold or cell culture or tissue after application of electric or magnetic field.

The inventive method is more effective than conventional methods of electroporation, at least because the cells in proximity to the scaffold are also in proximity to the substance (e.g., nucleic acids, proteins, small molecules, nanoparticles, and the like) to be taken into the cell. Furthermore, some cells that are adherent to the scaffold or associated with the scaffold or embedded into the scaffold receive survival signals from scaffold proteins, thereby improving cell viability during and after the electroporation.

Once the electroporation is completed, the cells can be released from the scaffold by using a variety of agents, such as calcium free medium ±calcium chelators such as EDTA or EGTA, or inhibitors of cell adhesion, or enzymes that degrade the biocompatible materials of the scaffold such as the extracellular matrix; and/or by using gentle fluid flow.

Alternatively, after electroporation, the biocompatible scaffold in which transfected cells are associated, attached or embedded can be used directly in treatment. For example, the transfected cellularized scaffold may be useful in the treatment of wounds or as a delivery device for the transfected cells.

In some embodiments, the present disclosure provides a device for electroporation or magnetoporation, comprising: a biocompatible scaffold that promotes cellular adhesion to the scaffold or association with the biocompatible scaffold or embedding in the scaffold; and a distribution of nanoparticles, nanowires, or any combination thereof, wherein the distribution is at least partially encapsulated into the biocompatible scaffold and the nanoparticles or nanowires are either electrically conductive or magnetically responsive or both, wherein the distribution of nanoparticles is configured to produce an electric or magnetic field in a physiological solution, media, blood, plasma, serum, extracellular or intercellular fluid, and/or mammalian tissue, for electroporation or magnetoporation, respectively, in response to at least one of the following: a) electrically coupling the distribution to an electrical power source or b) disposing the distribution in an electric or magnetic field.

In other embodiments, a device for electroporation, comprising: a biocompatible scaffold comprising a biopolymer that promotes cellular adhesion or association or embedding to the biocompatible scaffold; and a distribution of nanoparticles, nanowires, or any combination thereof, wherein the distribution is at least partially encapsulated into the biocompatible scaffold and the nanoparticles or nanowires are either electrically conductive or magnetically responsive or both, wherein the distribution comprises i) nanoparticles or nanowires made of a first material and ii) nanoparticles and nanowires made of a second material, wherein the first and second materials have different electron affinities, wherein the distribution, when in contact with a conductive fluid, is configured to produce an electric field for electroporation.

Additionally, an electroporation system is disclosed wherein a device as described above is provided and further comprising an electrode; and an electrical power source couplable to the device and the electrode to generate an electrical field between the device and the second electrode. In alternative embodiments, instead of an electrical power source, a magnetic field source is provided and configured for placement near the device and for generating a magnetic field that results in the device generating an electrical field or magnetic field in response to the magnetic field generated by the magnetic field source.

In a further embodiment, a method for delivery of delivery material to cells is provided, the method comprising: adhering, or embedding, or associating the cells to the device with a delivery material disposed in, or receivable into, the biocompatible scaffold; generating an electric or magnetic field using the device; and delivering the delivery material from, or through, the biocompatible scaffold into the adhered or embedded or associated cells during or after the generating of the electric or magnetic field.

Further provided is a method for treating an organ or tissue of a mammalian subject, the method comprising: applying the device of claim 1 to the tissue of the subject to contact cells of the tissue with the device, wherein the device further comprises a delivery material for treating the tissue of the subject disposed in, or receivable into, the biocompatible scaffold; generating the electric or magnetic field using the device; and delivering the delivery material into the contacting cells during or after the generating of the electric or magnetic field.

Additionally, the present disclosure provides a method for treating cancer of a mammalian subject, the method comprising: applying the device of claim 1 to the subject to contact cells of the subject with the device, wherein the device further comprises a delivery material for treating the cells of the subject disposed in, or receivable into, the biocompatible scaffold; generating the electric or magnetic field using the device; and delivering the delivery material from the device into the contacting cells during or after the generating of the electric or magnetic field.

### BRIEF DESCRIPTION OF THE DRAWINGS

The accompanying drawings, which are incorporated in and constitute a part of this specification, exemplify various embodiments of the present invention and, together with the description, serve to explain and illustrate principles of the present disclosure. The drawings are intended to illustrate major features of the exemplary embodiments in a diagrammatic manner. The drawings are not intended to depict every feature of actual embodiments nor relative dimensions of the depicted elements, and are not generally drawn to scale.

FIG. 1 is a schematic representation of one example of a conventional electroporation system and method;

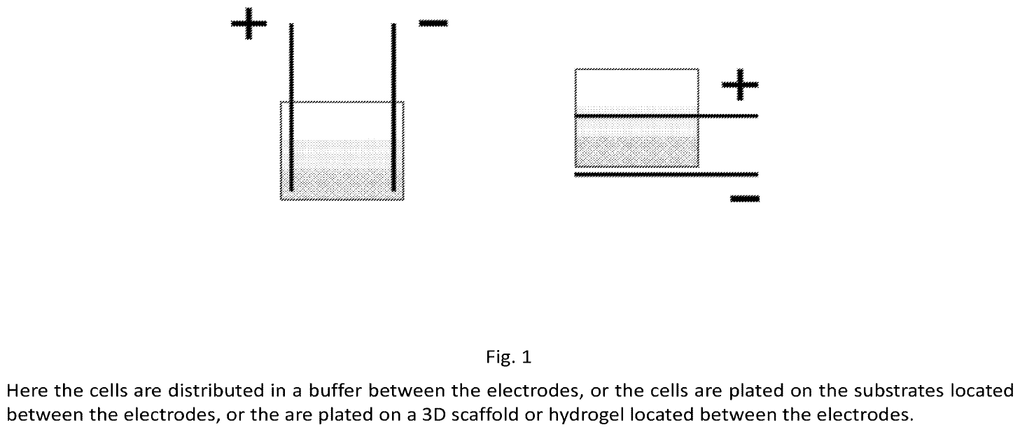

FIGS. 2A and 2B are schematic representations of a conventional electroporation system using a porous substrate;

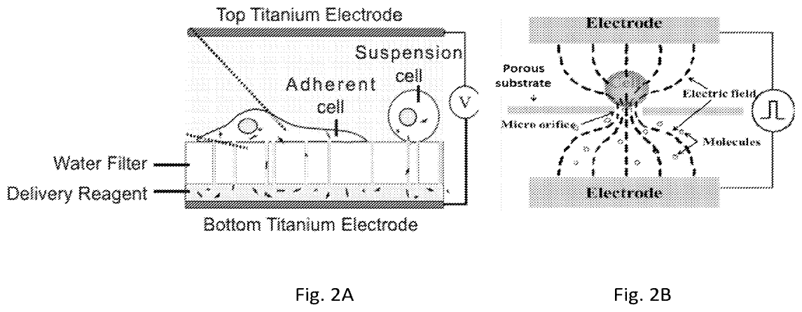

FIG. 3 is a schematic representation of a conventional electroporation system using patterned electrodes;

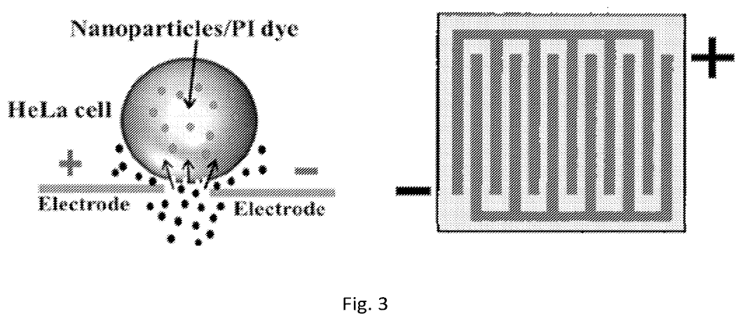

FIG. 4 illustrates an electroporation device according to embodiments of the present disclosure illustrating a biocompatible scaffold with a pulsed electric field applied;

FIG. 5 shows an electroporation device according to embodiments of the present disclosure illustrating a scaffold comprising a collagen membrane with an integrated silver nanowire network and showing generation of localized electric or magnetic field within the scaffold upon application of high intensity electric or magnetic pulses;

FIGS. 6A and 6B illustrate schematically an electroporation device according to embodiments of the present disclosure where the electroporation device is comprised of two elements: a porous sponge configured as a reservoir for nanoparticles or nucleic acids (6A), and a membrane including a patterned network of nanowires integrated in the membrane;

FIG. 7 illustrates an electrical closed loop (dash line) formed by connecting nanowires in three-dimensional structures according to embodiments of the present disclosure;

FIG. 8 is a cross-sectional view showing another example of collagen membrane including a silver nanowire mesh that electrically connects two different types of particles in a scaffold according to embodiments of the present disclosure;

FIGS. 9A and 9B schematically illustrate two embodiments of an electroporation system of the present disclosure including an electroporation device, mammalian cells and electrical power source;

FIG. 10 is a photograph showing alignment of HFF-1 adherent cells along collagen fibrils according to the present disclosure;

FIGS. 11A and 11B illustrate two different arrangements of electrodes and cell alignment according to embodiments of the present disclosure;

FIG. 12 shows GFP expression on two different collagen membranes according to the conditions of Experiment 1; and

FIG. 13 shows a comparison of GFP expression on two different collagen membranes with different electric field configurations, according to the conditions of Experiment 2.

### DETAILED DESCRIPTION

As described in detail below, embodiments of the present invention describe improved conductive devices and methods for electroporation over the current methods of cellular electroporation. In one embodiment, a device is provided that generally includes a biocompatible scaffold comprised of biocompatible material or membrane (e.g., collagen and/or other extracellular matrix, and the like) and incorporates a metal or polymer network or dispersion of conductive particles that conducts an electrical current through the scaffold. The biocompatible material or membrane that makes up the scaffold is configured to adsorb or trap or bind nucleic acids (e.g., RNA or DNA), nanoparticles, proteins and/or small molecules. Subsequently, cells are placed in proximity to or in contact with the scaffold and an electrical current is passed through the scaffold, thereby facilitating cellular entry of the nucleic acids, nanoparticles, proteins and/or small molecules, particularly into those cells that are in proximity of the scaffold.

The nanoparticles of the current invention include carbon-based nanoparticles, metal nanoparticles, ferroelectric nanoparticles, ferromagnetic nanoparticles, piezoelectric nanoparticles, piezomagnetic nanoparticles, ceramic nanoparticles, semiconductor nanoparticles, polymeric nanoparticles, lipid nanoparticles, silica nanoparticles, exosomes, leukosomes, or aposomes.

In some embodiments a device for electroporation or magnetoporation is disclosed, generally comprising: a biocompatible scaffold that promotes cellular adhesion to or association with the biocompatible scaffold; and a distribution of nanoparticles, nanowires, or any combination thereof, wherein the distribution is at least partially encapsulated into the biocompatible scaffold and the nanoparticles or nanowires are either electrically conductive or magnetically responsive or both, wherein the distribution is configured to produce an electric or magnetic field in a physiological solution, media, blood, plasma, serum, extracellular or intercellular fluid, and/or mammalian tissue, for electroporation or magnetoporation, While many of the embodiments are described as electroporation devices, it should be understood that the devices are also suitable as magnetoporation devices.

The inventors have invented biocompatible scaffolds, such as the fibrillar Nanoweave® biopolymer, which can be further developed to create electroporation devices. Examples of biocompatible scaffolds which may be employed in an electroporation device include, but are not limited to, those described in U.S. Pat. Nos. 9,724,308 and 10,065,046, both of which are incorporated herein by reference in their entireties. In some embodiments, the present disclosure provides new fibrillar collagen materials that incorporate metal and ferroelectric nanoparticles into its structure. Examples of several types of biocompatible scaffolds for electroporation according to the present disclosure are described in detail below.

For purposes of the present disclosure, “biopolymer” is defined as a biocompatible and biodegradable material selected from the group consisting of collagen, fibronectin, fibrin, laminin, elastin, hyaluronic acid, chitosan, silk, peptides, biodegradable block copolymers, lactide and glycolide polymers, caprolactone polymers, hydroxybutyric acids, polyanhydrides and polyesters, polyphosphazenes, polyphosphoesters, poly(ethylene glycol) (PEG) and poly(ethylene oxide) (PEO) (including PEG and PEO with different end-functionalities, as well as bifunctional cross-linkers or crosslinking agents), or any combination thereof.

In one embodiment, an electroporation device includes a collagen scaffold (also sometimes referred to herein interchangeably as a membrane) with an incorporated mesh of silver nanowires. The collagen scaffold is comprised of a material that may be configured to have tunable conductivity from 10 Ω/sq to 1000 Ω/sq to non-conductive (below the percolation threshold), tunable thickness of the membrane from 1 μm to 20 μm, and tunable degradation from 4 weeks to 1 year. In one example, this material is comprised of Nanoweave® biopolymer (available from Fibralign Corporation, Union City, Ca) This material can be used for electroporation to deliver nucleic acids, nanoparticles, proteins and/or small molecules to the cells plated on the scaffold in the following configurations:

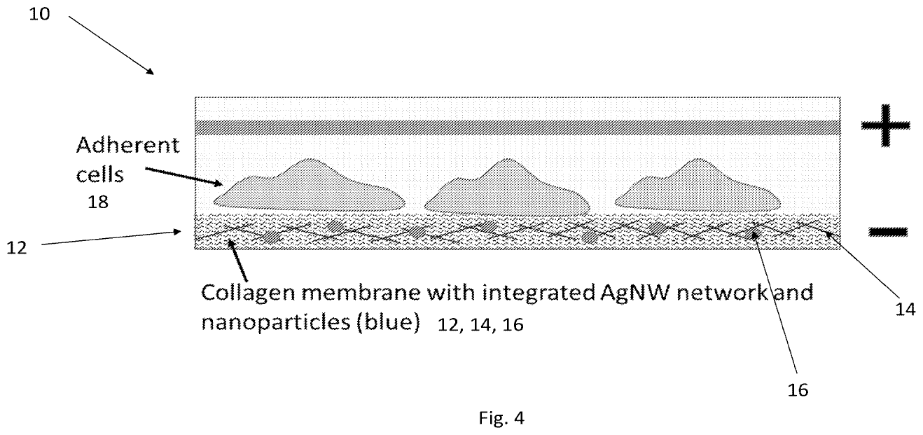

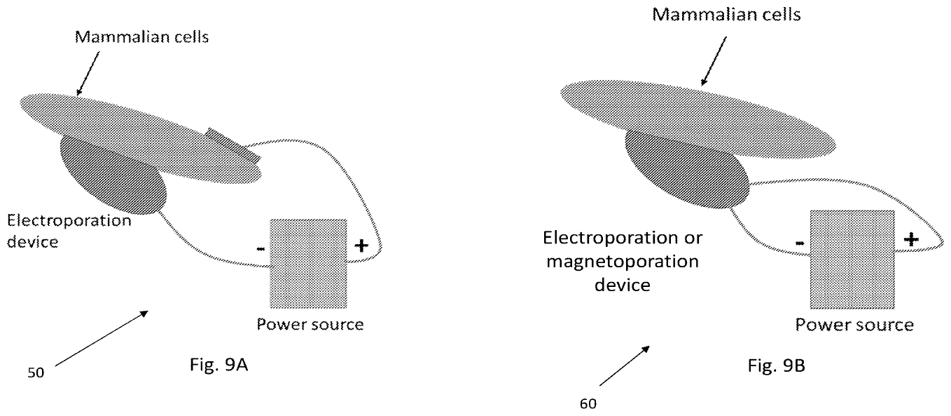

Turning to FIG. 4 an electroporation device according to some embodiments of the present disclosure is shown. The device 10 includes a collagen scaffold or membrane 12, the collagen membrane having a silver nanowire (AgNW) network 14 and silver nanoparticles 16 integrated throughout the membrane 12. Cells 18 are adherent on the membrane 12. It is important to note that FIG. 4 (among others) is a simplified drawing of the membrane or scaffold, and that the device will typically be comprised of a three-dimensional scaffold structure of collagen membranes. While the cells are shown adhering to a top surface of the membrane for simplicity in FIG. 4, it should be understood that the cells may adhere to multiple surfaces of the membrane. An electrical power source (see FIGS. 9A and 9B) is coupled to membrane and the cells to create a pulsed electric field.

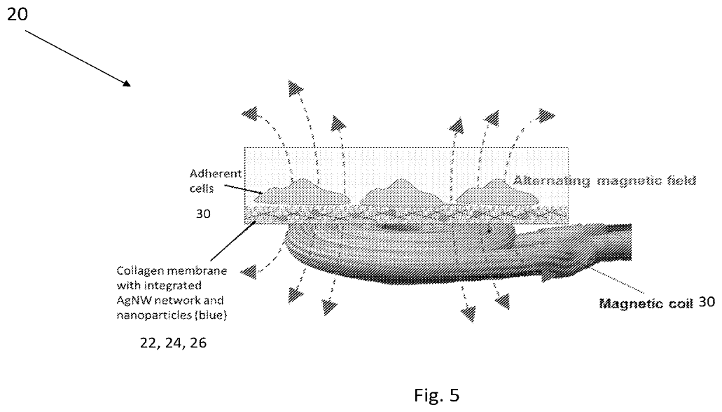

FIG. 5 shows an electroporation device according to another embodiment. In this embodiment the electroporation device 20 includes a collagen scaffold or membrane 22, the collagen membrane having a silver nanowire (AgNW) network 24 and silver nanoparticles 26 integrated throughout the membrane 22. Cells 28 are adherent to the membrane 22. In the embodiment, a magnetic coil 30 is applied to the device to create a high intensity pulsed magnetic field (0.05 T-5 T) which induces a localized electric or magnetic field in the scaffold sufficient for electroporation or magnetoporation of the cells plated on the scaffold. In some embodiments, patterned conductive nanowires or sub-percolation concentrations of silver nanowires are incorporated into the scaffold 22. Ferroelectric nanoparticle/nanowires can used as well.

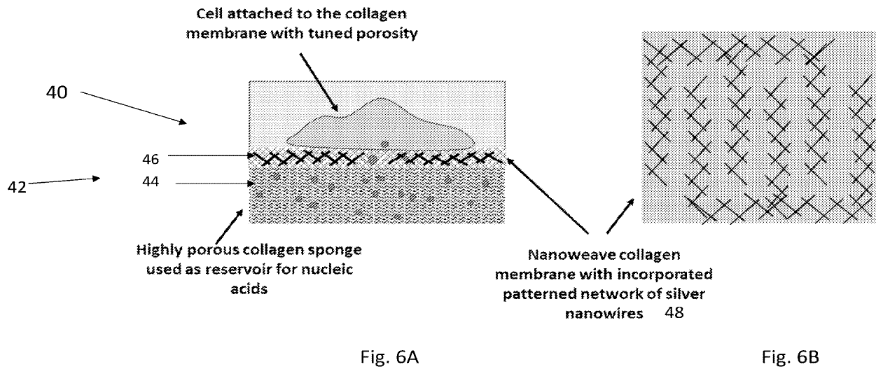

FIG. 6A illustrates another embodiment of an electroporation device 40, where the scaffold 42 is comprised of two elements: a porous sponge 44 and a collagen membrane 46 disposed atop the porous sponge. The porous sponge 44 is configured as a reservoir for nanoparticles or nucleic acids. The porous sponge generally has a porosity in the range of 50% to 95% and more particularly in the range of 80% to 92%. In this example, the collagen membrane 46 is a nanoweave collagen membrane with a patterned network of silver nanowires 48 incorporated in the membrane as shown in FIG. 6B. Of particular advantage, the network of silver nanowires may be patterned to allow for electroporation at the interface between two patterned lines, where the nanowire lines are connected to + and − of a power supply to apply an electrical pulse and to enable a selective transport of the nanoparticles and/or nucleic acids.

Each of the electroporation devices described above can be modified using other extracellular matrix components in addition to collagen (e.g., fibronectin, and the like) and/or growth factors and/or small molecules that enhance viability of the electroporated cells, and/or their incorporation and growth in within the scaffold.

### Topical Application for Treatment of Skin Diseases or for Cosmetic Purposes.

The scaffolds with incorporated metal or/and ferroelectric nanoparticles can be used for these applications. Pulsed electric or magnetic field can be applied to skin covered by the scaffolds described above to facilitate electroporation into the skin. Drug or nanoparticles or nucleic acids can be applied to the skin or supplied by the scaffold or injected into the skin. These materials can be incorporated into electroporated cells.

### Minimally Invasive or Invasive Treatments for Diseased Organs or Tissues.

The scaffold may be delivered to tissues or organs in need of treatment using minimally invasive techniques (e.g., laparoscopy) or robotic surgery; or with traditional open surgical approaches. Applications may be useful for any condition treated with minimally invasive or robotic surgery or open surgery approach. For example, the implantable scaffold with conductive/ferroelectric nanoparticles and treatment drug can be delivered in form of hydrogel or microparticles to the cancer area and then activated by external magnetic field. The treatment drug will be delivered into the cells in the cancer area by electroporation. As an example, an irreversible electroporation can be used as non-thermal ablative method for treatment of prostate and renal cancer.

### Enhancing Cellular Therapies.

The scaffold can be used for electroporation of any mammalian cell type. The scaffold could be used for electroporating small or large batches of cells in solution, flowing through the scaffold, or adhering to or embedding in or associating with the scaffold. An example of an application would be the use of the scaffold to transfect T lymphocytes with mRNA encoding a chimeric antigen receptor [CAR-T cell]. CAR-T cells have been used in the treatment of cancer.

### Regenerative Medicine Applications

The implantable scaffold may be used for wound or bone healing, cartilage regeneration, tendon repair, reconstruction of soft tissues such as breast reconstruction, regeneration of cardiac or skeletal muscle; repair of hollow conduits such as blood vessels, ureters and bladder, trachea and airways, esophagus and other portions of the GI tract. It can be used for strengthening of subcutaneous tissue, for example, to treat cellulite.

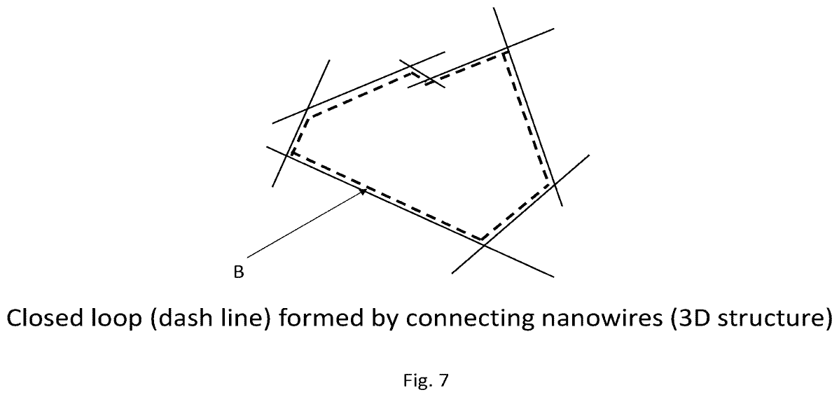

When using an implantable scaffold for healing or regeneration, a closed loop is formed by connecting nanowires as shown in FIG. 7. A closed loop is defined as having a diameter, the diameter of a closed loop (shown as lines B in FIG. 7) of connected nanowires is the diameter of the minimum sphere containing the loop. Alternatively, it can be measured as a maximum Feret's diameter of the loop.

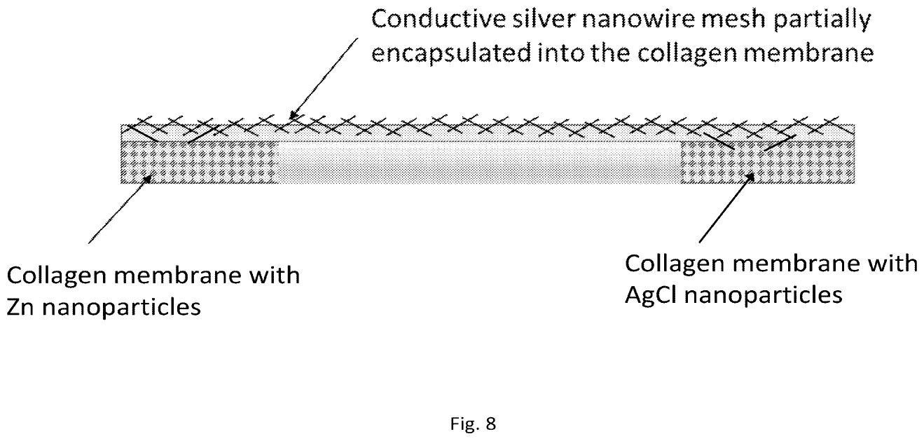

To create a device that can generate an ion current capable of inducing charged species to migrate in a desired direction, a scaffold comprised of fibrillar collagen membrane (e.g., made of type 1 atelocollagen) with regions of percolating Zn nanoparticles and regions of percolating silver chloride particles can be printed on a substrate, with a conductive silver nanowire network mesh (or an oxidized silver nanowire network) partially encapsuled into the collagen membrane as illustrated in FIG. 8. This provides a porous collagen scaffold with regions percolating Zn nanoparticles and silver chloride particles that are electrically connected to AgNWs (silver nanowires) that is biocompatible and biodegradable. Collagen promotes cell adhesion. In order to induce a spontaneous electric current in the scaffold this scaffold should be placed into a physiological solution (saline, PBS, cell culture media, wound exudate, extracellular fluid) such that the Zn nanoparticles, silver chloride particles, and AgNWs are connected by a conductive extracellular matrix or cellular clusters/aggregates or conductive tissue. Once in contact with physiological fluid containing ions, electrons will flow from the Zn particles (anode) through the silver nanowire network to the silver chloride particles (cathode). This electron flow will be counter-balanced by a corresponding ionic fluid in the surrounding media, which initiates the migration of charged species and precondition or facilitate or enhance transfection of introduced nucleic acid into the cells located in the media.

For example, percolating regions of Zn nanoparticles, (or nanowires), silver chloride particles (or nanowires) and Ag nanowires (or nanoparticles) are connected by a mammalian tissue (e.g., skin) and the matrix (or scaffold) containing the Zn, silver chloride, and Ag nanowires or nanoparticles is filled with a media conducting ions (e.g., forming a “salt bridge”).

It is also known that Zn—Ag system is highly antibacterial. (see Fan W, Sun Q, Li Y, Tay F R, Fan B. Synergistic mechanism of Ag+-Zn2+ in anti-bacterial activity against Enterococcus faecalis and its application against dentin infection. J Nanobiotechnology. 2018 Jan. 31; 16(410).

The inventors have discovered using the induced electric field for electroporation. If drug or mRNA are present in the vicinity of scaffold, then they can be delivered into the cells contacting the scaffold via electroporation. FIGS. 9A and 9B illustrate examples of an electroporation device 50 (FIG. 9A) and an electroporation or magnetoporation device (FIG. 9B) 60. The electroporation device generates high electrical pulses while magnetoporation device generates high magnetic pulses. The present metal nanowires (e.g., silver nanowires) substantially enhance the electromagnetic field in the vicinity of the conductive nanowires due to their high aspect ratios and, specifically, in the vicinity of the nanowire's tips. Alternatively, magnetic field induces Foucault's currents (or Eddy currents) that generate the secondary localized magnetic field which enables enhanced magnetoporation.

Magnetoporation has been successfully demonstrated for delivery of drugs and nucleic acids into cells but it requires extremely high magnetic field 1 T-7 T. The inventors have discovered that conductive nanowires dramatically reduce the intensity of magnetic field needed for an effective magnetoporation. The relatively small intensity pulse magnetic field (less than 0.3 T) is sufficient for efficient magnetoporation due to an enhancement of electromagnetic field near the ends of conductive (silver) nanowires.

As described above and further described below, in some embodiments the biocompatible scaffold comprises a biopolymer that promotes cellular adhesion to the biocompatible scaffold. The scaffold or member may further include a distribution of nanoparticles. In some embodiments the distribution of nanowires forms a conductive nanowire network. In one example, the conductive nanowire network does not have any closed loops of connected nanowires with a diameter of more than 200 micrometers. Alternatively, the conductive nanowire network does not have any closed loops of connected nanowires with a Feret's diameter of more than 100 micrometers in at least one direction. The conductive nanowire network may be configured for coupling to the electrical power source.

In another example, the conductive nanowire network comprises a first nanowire network and a second nanowire network, wherein the first and second nanowire networks are not conductively coupled and are configured for independent coupling to the electrical power source. Preferably, but not necessarily, for each point of the first nanowire network, a distance between the point and a nearest point of the second nanowire network is no more than 100 micrometers.

In some embodiments the conductive nanowire network has a conductivity at least 0.5 S/cm. In some embodiments, the conductive nanowire network has a sheet resistance of no more than 10000 ohm/sq. Preferably, a distance from any point on the surface of the biocompatible scaffold to a nearest one of nanoparticles or nanowires is no more than 100 micrometers. In some embodiments, the nanoparticles are selected from the group consisting of carbon-based nanoparticles, metal and metal oxide nanoparticles, ferroelectric nanoparticles, ferromagnetic nanoparticles, piezoelectric nanoparticles, or piezomagnetic nanoparticles. Additional nanoparticles may also be used, such as for example ceramic nanoparticles, semiconductor nanoparticles, polymeric nanoparticles, lipid nanoparticles, silica nanoparticles, exosomes, leukosomes, or aposomes, and the like.

Generally, the scaffold can be configured to generate an electrical field in response to application of a pulsed magnetic field to the scaffold.

In another aspect, a delivery material is disposed within the biocompatible scaffold, or in the vicinity of the biocompatible scaffold, or in a reservoir attached to the biocompatible scaffold, and configured for delivery to adhered or associated or embedded cells during production of the electric or magnetic field using the device. As shown in FIG. 6A a porous matrix may be attached to the biocompatible scaffold and configured to act as the reservoir for the delivery material. In some embodiments, the delivery material is selected from at least one of the following: nucleic acids; proteins; nanoparticles; or drugs

The scaffold or member are typically comprised of biocompatible and biodegradable materials selected from the group consisting of collagen, fibronectin, fibrin, laminin, elastin, hyaluronic acid, chitosan, silk, peptides, biodegradable block copolymers, lactide and glycolide polymers, caprolactone polymers, hydroxybutyric acids, polyanhydrides and polyesters, polyphosphazenes, polyphosphoesters, poly(ethylene glycol) (PEG) and poly(ethylene oxide) (PEO) (including PEG and PEO with different end-functionalities, as well as bifunctional cross-linkers or crosslinking agents), or any combination thereof.

In a further aspect, the device of the present disclosure is configured to facilitate flow through the biocompatible scaffold of the blood, plasma, serum, extracellular fluid, buffer or medium in which the mammalian cells or extracellular vesicles are placed.

The present disclosure also provides methods for delivery of delivery material to cells is provided, which generally comprise adhering or associating or embedding the cells to the scaffold with a delivery material disposed in, or receivable into, the biocompatible scaffold; generating an electric or magnetic field using the device; and delivering the delivery material from, or through, the biocompatible scaffold into the adhered or embedded or associated cells during the generating of the electric or magnetic field. To generate the electric or magnetic field the device and an additional electrode are coupled to a power source to generate an electric field between the device and the additional electrode.

Alternatively, an electric or magnetic field are generated by positioning a magnetic field source near the device, applying a magnetic field from the magnetic field source to the device, and generating the electric or magnetic field by the device in response to the applied magnetic field.

In one embodiment, the method is carried out with a device that includes a porous biopolymer matrix attached to biocompatible scaffold, and wherein the porous biopolymer matrix comprises the reservoir of delivery material. In some embodiments, the method further comprises drawing the delivery material from the porous biopolymer matrix into the device for delivery to the adhered cells. Optionally, the adhered cells are released after delivery of the delivery material.

### EXAMPLES

The following examples are provided for illustration purposes only and are not intended to limit the scope of the inventions in any way.

The following experiments were carried out to test the effect of nanowires on electroporation-induced transfection of HFF-1 fibroblast cells adhering to a collagen membrane. Collagen membranes were manufactured with and without imbedded silver nanowires (AgNW) present on one surface of the collagen. To create membranes with no nanowires a solution of monomeric molecular collagen was coated, dried, and removed from a cyclic olefic polymer (COP) substrate. Collagen membranes containing AgNW were created by first coating AgNW roughly 25 nanometers in diameter and 10 microns long on a COP substrate using a 100 ppm AgNW dispersion. The molecular collagen solution was then coated on top of the layer of dried AgNW. When the collagen membrane is removed from the substrate the nanowires remain imbedded on the surface layer of the collagen. In both cases the membrane surface which was originally face down on the substrate was placed face up and used for incubation of the adherent cells, meaning the AgNW, if present, were in close proximity to the cells.

All membranes were then secured at the bottom of a self-adhesive silicone well (manufactured by Ibidi) and sterilized. HFF-1 cells were seeded at 100 000 cells/well (day 0), and transfected with GFP mRNA on day 1 using electroporation (EP). The EP protocol included a high voltage (HV) pulse sequence (two 250 V, 5 ms duration, in both forward and reverse directions) followed by a low voltage (LV) pulse sequence (two sets of five 30 mV, 50 ms pulse at 100 ms interval). The adherent electrode used was the Nepa Gene CUY900-13-3-5 Electrode, and the pulse generator was BTX Harvard Electro Square Porator. The mRNA concentration was 20 ug/ml in an iso-osmolar buffer (iso-B), 400 ul of which was added for the EP procedure. On days 1 and 2 post-transfection, cells were monitored for GFP expression using a fluorescent microscope. On day 2, cell lysates were prepared for quantitation by GFP ELISA.

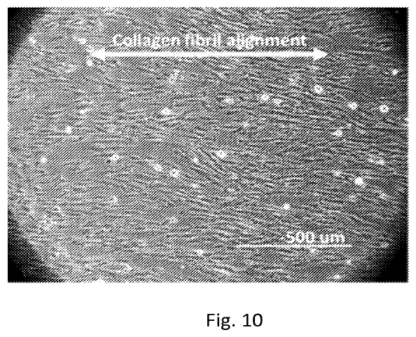

The HFF-1 cells had a tendency to align in the direction of the aligned collagen fibrils, as shown in FIG. 10. Two experiments were conducted according to the protocol above, as follows:

### Experiment 1

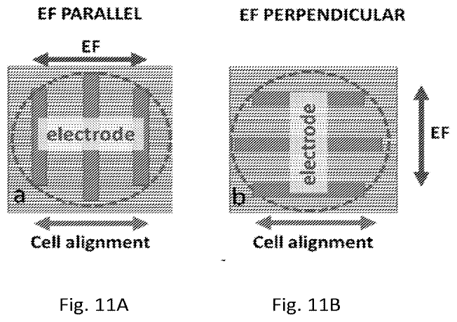

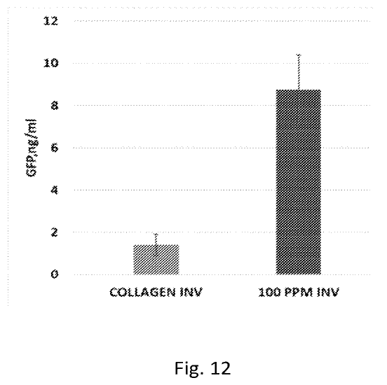

As shown in FIGS. 11A and 11B, three electrodes are used, with a middle electrode having high voltage and two outer electrodes are at lower voltage. The electric field of the electroporation pulse could be applied either parallel (FIG. 11A) or perpendicular (FIG. 11B) to the alignment of the cells. In Experiment 1 the electric field of the EP pulses was applied parallel to the alignment of the cells on the collagen membranes. The results of the ELISA measurements made on these samples are shown in FIG. 12, comparing the GFP measured for cells on collagen membranes both with and without nanowires. It is seen that the transfection of the cells was approximately a factor five higher for the membrane containing silver nanowires, which is labeled as 100 PPM INV, as compared to a collagen only membrane.

### Experiment 2

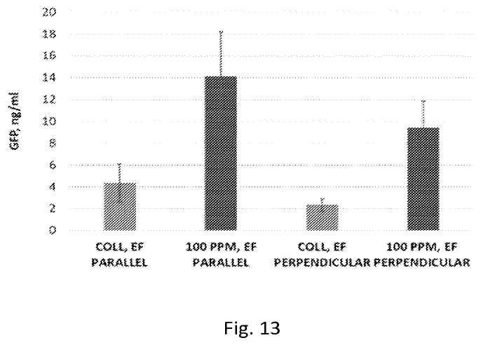

In Experiment 2, the electric field of the EP pulse was applied both parallel and perpendicular to the cell alignment (FIG. 11B) for membranes with and without nanowires. Again, as shown in FIG. 13 the transfection was significantly higher in the films containing nanowires, regardless of the orientation of the electrodes. The GFP expression on the 100 ppm AgNW collagen membrane was significantly higher as compared to that on the collagen-only membrane, for both parallel (3.3-fold) and perpendicular (4.1-fold) EF configurations.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A device for electroporation or magnetoporation, comprising:
a biocompatible scaffold that promotes cellular adhesion to the biocompatible scaffold or cellular association with the biocompatible scaffold or cellular embedding in the biocompatible scaffold; and
a distribution of nanoparticles, nanowires, or any combination thereof, wherein the distribution is at least partially encapsulated into the biocompatible scaffold and the nanoparticles or nanowires are either electrically conductive or magnetically responsive or both, wherein the distribution of nanoparticles is configured to produce an electric or magnetic field in a physiological solution, media, blood, plasma, serum, extracellular or intercellular fluid, and/or mammalian tissue, for electroporation or magnetoporation, respectively, in response to at least one of the following: a) electrically coupling the distribution to an electrical power source or b) disposing the distribution in an electric or magnetic field.

2. The device of claim 1, wherein the biocompatible scaffold comprises a biopolymer that promotes cellular adhesion to the biocompatible scaffold.

3. The device of claim 1, wherein the biocompatible scaffold comprises an aligned collagen membrane.

4. The device of claim 1, where the biocompatible scaffold is modified by incorporating ligands such as antibodies or aptamers to enhance the adherence of cells or cell subsets.

5. The device of claim 1, wherein the distribution of nanoparticles comprises nanowires above the percolation threshold.

6. The device of claim 1, wherein the distribution of nanoparticles comprises a conductive nanowire network.

7. The device of claim 3 or claim 4 or claim 5, wherein the conductive nanowire network does not have any closed loops of connected nanowires with a diameter of more than 200 micrometers that does not have nanowires inside the loop.

8. The device of claim 6, wherein the conductive nanowire network does not have any closed loops of connected nanowires with a Feret's diameter of more than 100 micrometers in at least one direction.

9. The device of claim 6, wherein the conductive nanowire network is configured for coupling to the electrical power source.

10. The device of claim 6, wherein the conductive nanowire network comprises a first nanowire network and a second nanowire network, wherein the first and second nanowire networks are not conductively coupled and are configured for independent coupling to the electrical power source.

11. The device of claim 10, wherein, for each point of the first nanowire network, a distance between the point and a nearest point of the second nanowire network is no more than 100 micrometers.

12. The device of claim 6, wherein the conductive nanowire network has a resistance between 1-10,000 ohm/sq

13. The device of claim 1, wherein a distance from any point of the biocompatible scaffold to a nearest one of nanoparticles or nanowires is no more than 100 micrometers.

14. The device of claim 1, wherein the device is configured to generate an electrical field in response to application of a pulsed magnetic field to the device.

15. The device of claim 1, further comprising a delivery material disposed within the biocompatible scaffold, or in the vicinity of the biocompatible scaffold, or in a reservoir attached to the biocompatible scaffold, and configured for delivery to adhered or associated or embedded cells during or after application of the electric or magnetic field using the device.

16. The device of claim 15, further comprising a porous matrix attached to the biocompatible scaffold and configured to act as the reservoir for the delivery material.

17. The device of claim 1, wherein the biopolymer comprises biocompatible and biodegradable materials selected from the group consisting of collagen, fibronectin, fibrin, laminin, elastin, hyaluronic acid, chitosan, silk, peptides, biodegradable block copolymers, lactide and glycolide polymers, caprolactone polymers, hydroxybutyric acids, polyanhydrides and polyesters, polyphosphazenes, polyphosphoesters, poly(ethylene glycol) (PEG) and poly(ethylene oxide) (PEO) (including PEG and PEO with different end-functionalities, as well as bifunctional cross-linkers or crosslinking agents), or any combination thereof.

18. The device of claim 1, wherein the delivery material is selected from at least one of the following: nucleic acids; proteins; nanoparticles; or drugs.

19. The device of claim 1, wherein the nanoparticles are selected from the group consisting of carbon-based nanoparticles, metal nanoparticles, metal oxide nanoparticles, ferroelectric nanoparticles, ferromagnetic nanoparticles, piezoelectric nanoparticles, or piezomagnetic nanoparticles.

20. The device of claim 19, further comprising additional nanoparticles selected form the group consisting of ceramic nanoparticles, semiconductor nanoparticles, polymeric nanoparticles, lipid nanoparticles, silica nanoparticles, exosomes, leukosomes, or aposomes.

21. The device of claim 1, wherein the device is configured to facilitate flow through the biocompatible scaffold of the blood, plasma, serum, extracellular fluid, buffer or medium in which the mammalian cells or extracellular vesicles are placed.

22. A device for electroporation, comprising:
a biocompatible scaffold comprising a biopolymer that promotes cellular adhesion to the biocompatible scaffold or cellular association with the biocompatible scaffold or cellular embedding in the biocompatible scaffold; and
a distribution of nanoparticles, nanowires, or any combination thereof, wherein the distribution is at least partially encapsulated into the biocompatible scaffold and the nanoparticles or nanowires are either electrically conductive or magnetically responsive or both, wherein the distribution comprises nanoparticles or nanowires made from two or more materials, each having a different electron affinity, wherein the distributions of the materials with different electron affinity are electrically connected and, when in contact with an ionic fluid, are configured to produce an ionic current to facilitate or enhance electroporation.

23. An electroporation system, comprising:
the device of claim 1;
an electrode; and
the electrical power source couplable to the device and the electrode to generate an electrical field between the device and the second electrode.

24. A system, comprising:
the device of claim 1; and
a magnetic field source configured for placement near the device and for generating a magnetic field that results in the device generating an electrical field or magnetic field in response to the magnetic field generated by the magnetic field source.

25. A method for delivery of delivery material to cells, the method comprising:
adhering cells to the device of claim 1 or associating cells with the device of claim 1 or embedding cells in the device of claim 1 with a delivery material disposed in, or receivable into, the biocompatible scaffold;
generating the electric or magnetic field using the device; and
delivering the delivery material from, or through, the biocompatible scaffold into the adhered cells during the generating of the electric or magnetic field.

26. The method of claim 25, wherein generating the electric or magnetic field comprises coupling the device and an additional electrode to a power source and generating the electric field between the device and the additional electrode.

27. The method of claim 25, wherein generating the electric or magnetic field comprises positioning a magnetic field source near the device, applying a magnetic field from the magnetic field source to the device, and generating the electric or magnetic field by the device in response to the applied magnetic field.

28. The method of claim 25, wherein the device further comprises a porous biopolymer matrix attached to the biocompatible scaffold, wherein the porous biopolymer matrix comprises the reservoir of delivery material.

29. The method of claim 28, further comprising drawing the delivery material from the porous biopolymer matrix into the device for delivery to the adhered cells.

30. The method of claim 25, further comprising releasing the adhered cells after delivery of the delivery material.

31. A method for treating an organ or tissue of a mammalian subject, the method comprising:
applying the device of claim 1 to the tissue of the subject to contact cells of the tissue with the device, wherein the device further comprises a delivery material for treating the tissue of the subject disposed in, or receivable into, the biocompatible scaffold;
generating the electric or magnetic field using the device; and
delivering the delivery material into the contacting cells during the generating of the electric or magnetic field.

32. A method for treating cancer of a mammalian subject, the method comprising:
applying the device of claim 1 to the subject to contact cells of the subject with the device, wherein the device further comprises a delivery material for treating the cells of the subject disposed in, or receivable into, the biocompatible scaffold;
generating the electric or magnetic field using the device; and
delivering the delivery material from the device into the contacting cells during the generating of the electric or magnetic field.
